(19) United States
(12) Patent Application Publication
(10) Pub. No.: US 2026/0283781 A1
(43) Pub. Date: Sep. 24, 2026

(54) COMPRESSIBLE SOFT TISSUE PROSTHESES AND RELATED METHODS

(71) Applicant: Davol Inc., Warwick, RI (US)

(72) Inventors: Timothy John Butler, Pelham, NH (US); Skander Limem, Lynnfield, MA (US); Hannah S. Thompson, Chicago, IL (US); Martin Neff, Lauterbach (DE); Jacob T. Brennan, Wendell, NC (US)

(73) Assignee: Davol Inc., Warwick, RI (US)

(21) Appl. No.: 19/473,715

(22) PCT Filed: Apr. 12, 2024
(86) PCT No.: PCT/US2024/024180

§ 371 Date: Oct. 8, 2025

Related U.S. Application Data
(60) Provisional application No. 63/459,361, filed on Apr. 14, 2023.

(51) Int. Cl. A61F 2/12; A61F 2/00

(52) U.S. Cl. CPC A61F 2/12; A61F 2/0077; A61F 2210/0004; A61F 2240/001

(57) ABSTRACT

Implantable prostheses and related methods are generally described. In some embodiments, the prosthesis may be used for soft tissue reconstruction. The prosthesis may be formed of a three-dimensional, load-bearing porous scaffold with an interconnected pore network to enable tissue infiltration, and allow native tissue, blood vessels, and cells to colonize the scaffold. In some embodiments, the scaffold may be formed of a biodegradable material, such that it may degrade during the tissue infiltration process, leaving behind native tissue. In some embodiments, the scaffold may be formed of a triply period minimal surface, such as a gyroid, to enable high compressibility and high porosity. The compressibility of the implant may mimic the mechanical behavior of natural tissue, and allow the implant to be delivered through a small incision site (e.g., with an implant delivery funnel).

### CROSS-REFERENCE TO RELATED APPLICATIONS

This application claims the benefit under 35 U.S.C. § 119 (e) of U.S. Provisional Application 63/459,361, filed on Apr. 14, 2023, the content of which is incorporated herein by reference in its entirety.

### FIELD

The present disclosure relates to an implantable prosthesis, and more particularly to a prosthesis for reconstruction and/or augmentation of soft tissue, including a human breast.

### BACKGROUND

Breast reconstruction following mastectomy has become an integral and important part of breast cancer treatment with the surgery providing the patient with both aesthetic and psychosocial benefits. In the US, nearly 65% of breast reconstruction procedures now use a tissue expander to create a pocket for a permanent breast implant in the first step of the procedure. In some patients, a pocket for the breast implant can be formed without the use of a tissue expander. Once a pocket has been created, the tissue expander is removed, and replaced with a permanent breast implant in a second step.

Breast implants can also be used in breast augmentation and mastopexy procedures to augment breast size. In the latter procedure, a breast lift is combined with breast augmentation. Most commonly, the breast implant is placed in a pocket under the breast tissue, but in some cases, it is implanted under the chest wall.

Breast implants differ in dimensions, shape, and surface texture. A wide variety of different dimensions are available allowing the surgeon and patient to select from a range of projections, heights, widths and overall volume. In terms of shape, there are round and anatomically shaped implants, and the surfaces of the implants may be smooth, micro-textured or macro-textured. Generally, round implants have smooth surfaces, whereas anatomically shaped implants have dimpled micro- or macro-textured surfaces.

### SUMMARY

In some embodiments, an implantable soft tissue repair prosthesis includes a flexible porous scaffold formed of at least one triply periodic minimal surface, wherein the flexible porous scaffold is configured to recover at least 75% of its original volume upon a 50% compression in at least one direction.

In some embodiments, a method of implanting an implantable soft tissue prosthesis includes compressing the implantable prosthesis by at least 50% in at least one direction, the implantable prosthesis comprising a flexible porous scaffold formed of at least one triply periodic minimal surface, delivering the implantable prosthesis to an implant site, and recovering at least 90% of the compression in the at least one direction.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1A-1C are an isometric, side, and top view, respectively of a subunit of a scaffold of an implantable prosthesis according to some embodiments;

FIGS. 2A-2C are an isometric, side, and top view, respectively of a portion of a scaffold of an implantable prosthesis according to some embodiments;

FIG. 3 is a breast implant according to some embodiments;

FIGS. 4A-4B are breast implants according to other embodiments;

FIGS. 5A-5C are breast implants according to other embodiments with different spatial frequencies for the scaffold;

FIGS. 6A-6B are a top and side view of a breast implant, respectively, according to one embodiment;

FIGS. 7A-7B are a top and side view of a breast implant, respectively, according to another embodiment; and

FIGS. 8A-8B are a top and side view of a breast implant, respectively, according to another embodiment still.

### DETAILED DESCRIPTION

A growing number of patients considering breast reconstruction and augmentation are reluctant to have permanent breast implants placed in their breasts. This is particularly the case for women that have had a mastectomy and are now considering breast reconstruction. Some of these patients do not want to have a permanent foreign body placed in their breasts, and they do not want to run the risk of complications that may develop with permanent breast implants. The complications include potential capsular contraction, rupture or deflation of the implant, development of anaplastic large cell lymphoma (ALCL), infection, and migration of the implants causing asymmetry of the breasts. Many of these complications may need a procedure to correct, which may be costly and undesirable in addition to the accumulation of scar tissue and delayed healing.

In addition to the above, conventional breast implants (e.g., silicone-based implants) are typically formed of a uniform material with mechanical properties that do not accurately mimic natural soft tissue, which may include vasculature and variations in its stiffness and compressibility due to muscle and fat distribution. In particular, the Inventors have recognized that although conventional soft tissue implants are elastic, they often do not exhibit sufficient natural compressibility, allowing the implant to be compressed to a fraction of its original volume, and recover back to its original volume (or a fraction thereof), a behavior that is native to natural tissue. This lack of compressibility may also prevent the implants from being compressed during delivery, such that large incisions may be required for implantation. These incisions may enhance the risk of infection, prolong recovery, and form large undesirable scars on the patient.

In view of the above, the Inventors have recognized the benefits associated with implantable prostheses which balance mechanical properties to support surrounding anatomy with high rates of tissue infiltration which may facilitate an appropriate balance of mechanical support and feel. Thus, the Inventors have recognized the benefits associated with implantable prostheses that exhibit sufficient mechanical properties to support the natural tissue of the implant site and include sufficient void spaces to enable tissue ingrowth. In this way, the prostheses may provide sufficient mechanical support to the implant site while still enabling rapid tissue ingrowth. For example, in some embodiments, the implantable prostheses, which may be referred to as implants or prostheses, may achieve mechanical properties akin to natural tissue through the use of a load bearing, three-dimensional macroporous and flexible scaffold. The flexible scaffold may be formed of one or more types of subunits arranged in a repeating fashion. In some embodiments, the macroporous scaffold may include interconnected pores formed between the subunits. These pores may enhance the degradability of the scaffold by increasing the surface area, while also increasing the rates of tissue ingrowth. The size and arrangement of the subunits, as well as their material composition, may determine the mechanical properties of the prosthesis. For example, more densely arranged subunits (e.g., with a smaller spatial frequency or repeat distance between neighboring subunits) may result in a greater stiffness relative to less densely arranged subunits. It should be appreciated that in some embodiments, more densely arranged subunits may also result in greater material volume used in the prosthesis, which may increase the stiffness of the prosthesis, while a less densely arranged subunit scaffold may result in a less stiff prosthesis. Thus, the subunit properties may be adjusted to optimize the tissue infiltration rate as well as the compressibility of the implant.

In some embodiments, the load-bearing, three-dimensional macroporous and flexible scaffolds of the present disclosure may be at least partially formed of subunits formed of wavy walls. The lack of corners and straight lines may reduce stress concentrations within the implant and allow for greater compressibility. In some embodiments, the wavy walls may have planar symmetry curves to mimic natural soft tissue behavior, although other symmetrical properties are also contemplated. The subunits of the three-dimensional macroporous and flexible scaffold may be minimal surface structures that minimize their surface area by demonstrating zero mean curvature. The minimal surface structures may specifically be triply periodic minimal surfaces (TPMS). These minimal surfaces may result in a high surface area to volume ratio for the implant, which may enhance tissue ingrowth and implant degradation if the implant is formed of a degradable or bioresorbable material. The high surface area to volume ratio may also enhance the porosity of the implant while reducing the implant weight, which may in turn reduce the strain on the patient's implant site. The TPMS of the present disclosure may also enable tunable isotropic and/or anisotropic properties through the scaffold dependent upon their arrangement.

The TPMS subunits may be in the shape of gyroids, which in some embodiments may be approximated by the following trigonometric relationship relative to the three cardinal directions:

sin(x)cos(y)+sin(y)cos(z)+sin(z)cos(x)=0

Other TPMS structures, such as catenoids, helicoids, lidinoids, argyles, Bonnet family surfaces, Schwarz P surfaces, Schwarz D surfaces, hybrids thereof, and/or other TPMS structures are also contemplated. Such surfaces may reduce stress concentrations to improve the overall compressibility of the implants while increasing the porosity.

Depending on the shape, a scaffold of an implantable prosthesis may be compressible in both its radial and axial directions. Further, in some embodiments, the implant may be isotropic or anisotropic with respect to one or more mechanical properties. For example, in some embodiments, the implant may exhibit one or more mechanical properties in a first direction which may be within 10% to 20% of the mechanical properties in a second direction. Additionally or alternatively, in some embodiments, the implant may exhibit one or more mechanical properties in a first direction which may be only 2%, 5%, 10%, 20%, 30%, or 40% of the mechanical properties in a second direction. For example, in some embodiments, a compressive stiffness of the implant in a radial direction may be approximately 2%, 5%, 10%, 20%, 30%, or 40% of a compressive stiffness of the implant in an axial direction. Combinations of the foregoing are also contemplated. For example, in some embodiments, the radial compressive stiffness may be between or equal to about 2% and about 40% of the axial compressive stiffness, between or equal to about 5% and about 30% of the axial compressive stiffness, between or equal to about 10% and about 20% of the axial compressive stiffness, or between or equal to about 5% and about 20% of the axial compressive stiffness. Further, it will be appreciated that different mechanical properties may have different isotropic characteristics, such that a first mechanical property may be isotropic (e.g., where the first property in a first direction is within 10% or 20%, or within 10-20%, of the first property in a second direction) while a second mechanical property may be anisotropic (e.g., where the second property in a first direction is 2%, 5%, 10%, 20%, 30%, 40% or between 2% and 40% of the second property in a second direction). Additionally, the implants may be non-auxetic (e.g., exhibiting a positive Poisson's ratio) to better mimic natural tissue, but it should be appreciated that auxetic implants (e.g., exhibiting a negative Poisson's ratio) are also contemplated.

In some embodiments, the macroporous implant may include more than one type of subunit. For example, part of the implant scaffold may include gyroids while another part of the scaffold may include another minimal surface structure. It should be appreciated that alternative subunits may be used in conjunction with the TPMS subunits described above, including, but not limited to, enclosed cavities, partially-enclosed cavities, repeating unit cells or networks of unit cells, foam cell, Kelvin foam cell or other open-cell or closed-cell foam structures, crisscross structures, struts, ties, channels (whether open, closed or partially enclosed), waveguides, triangular structures, tetrahedron or other pyramid shape, cube, octahedron, octagon prism, icosidodecahedron, rhombic triacontahedron or other polyhedral shapes or modules (including Kelvin minimal surface tetrakaidecahedra, prisms or other polyhedral shapes), pentagon, hexagonal, octagon and other polygon structures or prisms, polygon mesh or other three-dimensional structure, honeycomb structures, tessellation structures such as Voronoi structures or others (which may comprise a pattern of shapes formed without overlaps or gaps between the shapes), and/or other appropriate structures. The three-dimensional implant may include a combination of the aforementioned three-dimensional subunit structures in an interconnected network.

In some embodiments, the flexible scaffolds of the implantable prostheses may have an architecture that provides a larger surface area and voids suitable to allow the scaffold to be colonized by cells and infiltrated by tissue, blood vessels, or combinations thereof following implantation. Accordingly, the implantable prostheses of the present disclosure may be characterized by an infill density, defined as the ratio of volume occupied by implant material in the scaffold divided by the total volume of the implant expressed as a percentage. The infill density may be designed to balance tissue infiltration, which may be maximized with reduced infill density, with sufficient compressive strength to support the surrounding tissue of the implant site.

The Inventors have also recognized that the weight of conventional permanent implants, which may range between 150 cc and 1200 cc, more commonly between 300 cc and 800 cc, are typically heavier than natural breast tissue of comparable size, and may therefore apply excess strain to the patient's anatomy, such as the chest wall. Such strain may result in discomfort and pain for the patient, and in some instances, can result in undesirable drooping or ptosis of the implant. In view of the above, the inventors have recognized the benefits associated with lightweight soft tissue implants, which may reduce the risk of patient discomfort and improve aesthetic outcomes. As described previously, the implants of the present disclosure may exhibit porosity characterized by an infill density. In some embodiments, the implants of the present disclosure may be approximately 80% to 97% lighter than conventional silicone implants at the time of implantation. In some embodiments, the infill density may also determine the overall weight of an implant at time of implantation. It should be appreciated that the porous scaffolds of the present disclosure may be generally lighter than conventional silicone implants, which may reduce the load on nearby tissue. In some embodiments, the implants may weigh less than 10 grams, although other weights are also contemplated, as the weight of an implant may depend upon a variety of parameters, including a desired application, material composition, geometry, and infill density, among others.

It should be appreciated that the overall weight of the implant may change over time due to material degradation, in contrast to conventional permanent implants. The gradual change in implant weight may allow for cellular infiltration, maturation, and cohesion (e.g., tissue formation). Following a prolonged period of time after implantation, the weight of the breast mound may be comparable to natural breast tissue, which in some embodiments may be less than the weight of a comparably sized permanent (e.g., formed of silicone) implant.

In some embodiments, an infill density of an implantable prosthesis according to any of the embodiments disclosed herein may be less than or equal to approximately 60% to induce tissue infiltration while still exhibiting desirable mechanical properties. In some embodiments, the implantable prostheses of the present disclosure may have an infill density greater than or equal to approximately 1%, 2%, 4%, 5%, 6%, 8%, 9%, 10%, 12%, 15%, 20%, 30%, 40%, 50%, combinations thereof, and/or any other suitable infill density. The implantable prostheses may also have infill densities less than or equal to approximately 60%, 50%, 40%, 30%, 20%, 15%, 12%, 10%, 9%, 8%, 6%, 5%, 4%, 2%, 1%, combinations thereof, and/or any other suitable infill density. Combinations of the foregoing, including, but not limited to, infill densities between approximately 1% and 60%, between approximately 3% and 15%, and between approximately 3% and 10% are contemplated. In some embodiments, the infill density may be between approximately 4% and 8%. It should be appreciated that the infill density of the implants may be dependent upon the geometry of the subunit(s) employed, the overall size of the implant, the method of fabrication, implant site needs, and/or any other appropriate parameter. Accordingly, the implants of the present disclosure may have any suitable infill density.

In some embodiments, the dimensions of the pores of the implant's scaffold may be large enough to allow needles to be inserted into the pores of the scaffold in order to deliver bioactive agents, cells, fat cells, and/or other appropriate compositions by injection. In some embodiments, the architecture of the scaffold is designed to allow needles with gauges of 12-21 to be inserted into the scaffold to allow for the delivery or loading of cells, tissue, collagen, bioactive agents and additives, including fat cells, using a syringe and without significantly damaging the scaffold and/or the delivered materials delivered into the scaffold. In some embodiments, the scaffolds may allow insertion of needles with outer diameters between or equal to 0.5 mm and 3 mm. Of course, depending upon the specific needs of the patient, the scaffold may be designed to accommodate needles of larger and smaller gauges, as the present disclosure is not so limited.

The scaffolds of the implantable prostheses may also be characterized by a weight per volume, which is determined by the density of the constituent material of the subunits, the size of the prosthesis, the infill density, and the number/size of subunits, among many other parameters. In some embodiments, the weight per volume of the scaffolds may be between approximately 0.01 and 0.2 g/cm³, between approximately 0.02 and 0.2 g/cm³, between approximately 0.03 and 0.05 g/cm³, between approximately 0.01 and 0.5 g/cm³, combinations thereof, and/or any other suitable weight per volume. Of course, weights per volume both less than and greater than those noted above are contemplated as the disclosure is not so limited.

A high surface area may increase the volume available for tissue infiltration throughout the scaffold. A high surface area may also increase the volume of therapeutic compounds that may be delivered with the implant (e.g., coated on the implant, as will be described in detail below). Thus, the scaffolds disclosed herein may also be characterized by an appropriately high surface areas in some embodiments. In some embodiments, the surface area of the scaffolds of an implant may be greater than or equal to approximately 500 cm², 1000 cm², 5000 cm², 1 m², 2 m², and/or any other suitable surface area. The surface area of the scaffolds may also be less than or equal to approximately 2 m², 1 m², 5000 cm², and/or any other appropriate surface area to ensure sufficient material available to support the surrounding tissue. Combinations of the foregoing are contemplated including, for example, areas that are between or equal to 500 cm² and 2 m², though other appropriate areas may also be used.

In some embodiments, the implant may be compressible such that it may be delivered to the implant site through a delivery apparatus, such as a Keller funnel. In some embodiments, the implant may be delivered into the breast through a funnel with a neck diameter (i.e., the narrowest part of the funnel) of approximately 2.5 to 7.5 cm, and more preferably 3.75 to 6.25 cm. In some embodiments, the implant may be delivered through a funnel with a neck diameter of 2.5 to 7.5 cm, and recover back to a percentage of the implant's original volume after delivery through the neck of the funnel into the implant site. In some embodiments, the implant may have a compressive stiffness that is anisotropic to facilitate delivery of the implant through a delivery apparatus and/or an incision. For example, an implant may have a compressive stiffness in a radial direction that is less than a compressive stiffness in an axial direction as described above, such that the base of the implant may be pinched together when passing through the neck diameter and/or the incision. However, it will be appreciated that isotropic embodiments may be suitable for delivery through a funnel as well.

In view of the above, the implant may therefore have a compressive modulus to enable its delivery to an implant site through an incision smaller than the original size of the implant to reduce the amount of scarring at the implant site for more rapid recovery and improved aesthetics. The compressive modulus may also allow the implant to be compressed when a compressive force is applied, and recover from compression when the compressive force is removed. The implant may be engineered so that the breast feels soft to the touch, similar to a natural breast. In some embodiments, the implant may allow the surgeon to restore or augment breast mass while maintaining or restoring the tactile sensation of the breast.

In some embodiments, the implant may have a compressive modulus between approximately 0.1 kPa to 10 MPa, 0.3 kPa to 1 MPa, and/or 3 kPa to 200 kPa, although other compressive moduli ranges both greater and less than those noted above are contemplated dependent upon the needs of the implant site tissue.

In some embodiments, the implant's compressibility may be characterized through a compression resilience, calculated as the work done during compression recovery divided by the work done during compression multiplied by 100. The compression resilience of the implants described herein may be between 1% and 80%, although other ranges, such as greater than or equal to approximately 50%, 60%, 70%, 80%, 90%, among others, are contemplated.

In some embodiments, the implant may be configured to recover at least 50% of its original volume upon the application and subsequent removal of a compressive force. Such behavior may enable easy delivery through an incision smaller than the size of the implant, and may also provide a more natural tactile feel to the implant. For example, following a 50% compression, the implants of the present disclosure may recover at least approximately 75% of their original volume. In some embodiments, the implants may recover at least greater than or equal to approximately 50%, 70%, 75%, 80%, 90%, 95%, 98%, and/or any other suitable proportion of its original volume upon the application and subsequent removal of the compressive force needed to compress the implant by 50%. In some embodiments, the implant may preferably recover at least approximately 90%, and more preferably 95% of its original volume after a 50% relative compression displacement in one or more directions. Depending on the embodiment, this recovery of the material after compression may be isotropic, though embodiments in which the recovery of the material is anisotropic (e.g., a radial compressibility and recovery being greater than or less than an axial compressibility and recovery) are contemplated.

In some embodiments, the implant may be characterized by a compressive modulus. The implant may have a compressive modulus that matches the native tissue for a more natural feel. In some embodiments, the implants may have a compressive modulus between approximately 0.01 kPa to 290 MPa, 0.1 kPa to 10 MPa, 0.1 kPa to 1 MPa, 0.1 kPa to 100 kPa, 2 kPa to 100 kPa, 0.1 kPa to 1 MPa, combinations thereof, and/or any other suitable compressive modulus for the application. For example, if the implant is used in breast reconstruction procedures, the compressive modulus may be between approximately 0.1 kPa to 1 MPa or between 2 kPa to 100 kPa. Embodiments with implant compressive moduli both greater and less than those noted above are contemplated.

It should be appreciated that the compressive modulus (and/or any other mechanical property) of the implant may vary over time as the implant degrades and is replaced by natural tissue. Accordingly, the aforementioned ranges in compressive moduli may refer to the implant prior to or at the time of implantation. In some embodiments, the compressive modulus of the implant may gradually decrease and approach the properties of native breast tissue, which may for example exhibit moduli between approximately 2 kPa to 70 kPa. The implant may approach the properties of native breast tissue in any suitable period of time following implantation, including, but not limited to, approximately 1 month to 5 years, approximately 3 months to 3 years, approximately 6 months to 2 years, and/or combinations thereof.

In some embodiments, the implantable prostheses may have one or more regions. In some embodiments, the one or more regions may differ in degradation rate and strength loss profile through any suitable means, including, but not limited to, regional variations in: infill density, constituent polymer molecular weight, fiber size, structural design, combinations thereof, and/or any other suitable means. For example, in some embodiments, the prosthesis may include one radial region formed of a first subunit arrangement and a second radial region formed of a second subunit arrangement having a greater infill density than the first region. Other exemplary embodiments will be described in detail below. In other embodiments, the implantable prostheses may have one or more gradients ranging between one or more subunit arrangements (e.g., size, distribution, infill density, etc.). For example, the implant may include a transition region between two regions to allow for a gradual change in properties. Thus, there may be a smooth (e.g., linear or non-linear, among others) transition between the properties that may be reliant upon the subunit arrangement, such as compressive stiffness. Embodiments including one or more distinct regions, as well as one or more gradient regions, are contemplated. Accordingly, the various scaffold and implant properties described herein may refer to the entire scaffold, or may refer to a portion of the scaffold.

Further to the above, in embodiments in which the various regions include differing properties, the implant may be characterized by an average property for the entire implant. For example, an average infill density may be calculated by multiplying the infill density of each region by the volume occupied by the region, summing the products for each region, and dividing by the number of regions. In some embodiments, an implant may have an average infill density greater than or equal to approximately 1%, 2%, 4%, 5%, 6%, 8%, 9%, 10%, 12%, 15%, 20%, 30%, 40%, 50%, combinations thereof, and/or any other suitable average infill density. The implantable prostheses may also have average infill densities less than or equal to approximately 60%, 50%, 40%, 30%, 20%, 15%, 12%, 10%, 9%, 8%, 6%, 5%, 4%, 2%, 1%, combinations thereof, and/or any other suitable average infill density. Combinations of the foregoing, including, but not limited to, average infill densities between approximately 1% and 60%, between approximately 3% and 15%, and between approximately 3% and 10% are contemplated. In some embodiments, an average infill density may be between or equal to 4% and 8%. It should be appreciated that the average infill density of the implants may be dependent upon the number of regions employed, the geometry of the subunit(s) employed in each region, the overall size of the implant, the method of fabrication, implant site needs, and/or any other appropriate parameter. Accordingly, the implants of the present disclosure may have any suitable average infill density.

As described earlier, in some embodiments, the implants of the present disclosure may be formed with anisotropic properties to better mimic the properties of natural tissue, which is typically non-homogenous and includes a variety of materials arranged in a precise fashion. Thus, the implants may have non-uniform properties across one or more directions of the implant, and may therefore be anisotropic in one or more ways (e.g., through mechanical or geometric properties, or combinations thereof). For example, the implants may have a first compression modulus in a radial direction, and a second different compression modulus in an axial direction. In some embodiments, the properties may vary in a gradient fashion in one or more directions of the implant. As described earlier, the scaffold may include one or more transition regions to enable a gradual change in properties between two regions in the implant. It should therefore be appreciated that the properties described herein may refer to either the entirety or a portion or portions of the implant.

In some embodiments, variations in mechanical properties such as compressive stiffness may be due to changes in geometric properties. For example, a smaller subunit with a smaller spatial frequency relative to other adjacent portions of the implant may result in a greater infill density, which may subsequently result in greater compressive stiffness relative to a scaffold with a larger subunit. Thus, the mechanical properties of the scaffold may be directly affected by the geometric properties of the scaffold. In some embodiments, variations in mechanical properties may be due to changes in constituent material properties of the subunits. For example, an implant can include two regions of subunits having substantially the same geometry, but made of different materials (and/or coated with different materials) to yield a difference in mechanical property. As will be described in greater detail below, in some embodiments, the implant may include support structures to provide local mechanical support to the implant.

In some embodiments, the implantable prostheses of the present disclosure may preferably have a generally dome-like shape to mimic the natural shape of the breast. In other embodiments, the implantable prosthesis may have a generally spherical or ellipsoidal shape to mimic the anatomical cavity left behind by a tissue removal procedure (e.g., lumpectomy), and/or any other natural or surgically formed cavity. However, it should be appreciated that the implantable prostheses of the present disclosure may have any suitable three-dimensional shape, including, but not limited to a sphere, dome, ellipsoid, hemisphere, cylinder, cone, dome, cuboid, tetrahedron, triangular or square prism, dodecahedron, torus, combinations thereof, and/or customized geometries. Partial versions of the aforementioned geometries are contemplated, for example, a half dome shape. Combinations of the aforementioned geometries are also contemplated, including, for example, a teardrop shape, a partial teardrop shape having a rounded shape at a first end portion and a tapered shape at a second end portion, or other appropriate shape. It should be appreciated that the aforementioned geometries, including partial versions and combinations thereof, may be selected and/or configured to mimic, augment, and/or otherwise achieve a desired anatomical shape, such as a desired shape of a natural breast or a portion of a natural breast. It should be appreciated that the term “ellipsoidal” as used herein refers to ellipsoidal three-dimensional shapes (which may have different average diameters in two or more directions), spheroidal shapes, and spherical shapes (which may have substantially similar average diameters in all directions). In some embodiments, the implantable prosthesis may be formed through an assembly of one or more scaffolds. For example, a spherical implant for use in a lumpectomy procedure may be formed by fusing or otherwise bonding together two hemispherical implants.

In some instances, the implantable prostheses disclosed herein may have non-symmetric shapes. For example, an implantable prosthesis may have different shapes in the front bottom and front top areas of the implant. The dimensions of the implant may be sized to augment breast tissue volume, to substitute for prior breast tissue volume, to change the volumetric distributions of breast tissue, to change the appearance of breast tissues, or to replace existing breast tissue volume with a smaller volume. The implantable prostheses may be sized or shaped to provide a low, moderate, or high-profile shape to the breast, wherein the implant profile determines the projection of the breast. High profile shaped implantable prostheses may be used to increase the height of the breast side wall, and provide patients with more upper pole fullness, or cleavage. Smaller increases in the height of the breast side wall may be obtained using implantable prostheses with low or moderate profile shapes. The implantable prostheses may be designed for use in the breast in sizes large enough to allow for their use in mastopexy and breast reconstruction. Patient-specific customized implant sizes are also contemplated. In some embodiments, the implantable prostheses may have a volume between 100 and 1200 cc (cubic centimeters), and/or a volume between 120 and 850 cc, though other volumes both greater and less than those noted above are also possible.

The implantable prostheses of the present disclosure may be formed of a biocompatible material which may promote rapid tissue or muscle in-growth into and around the prosthesis. In some embodiments, the implants may be formed of an absorbable material that may be replaced in vivo by the patient's natural tissue as the implant degrades.

In some embodiments, the prosthesis may be formed of an absorbable material (e.g., polymer or copolymer) that may be substantially resorbed after implantation within a 1 to 24-month timeframe, or 3 to 18-month timeframe, and retain some residual strength for at least 2 weeks to 6 months.

In some embodiments, the implant or scaffold may include an absorbable polymer comprising, or prepared from, one or more monomers selected from the group: glycolide, lactide, glycolic acid, lactic acid, 1,4-dioxanone, trimethylene carbonate, 3-hydroxybutyric acid, 3-hydroxybutyrate, 4-hydroxybutyric acid, 4-hydroxybutyrate, ε-caprolactone, 1,4-butanediol, 1,3-propane diol, ethylene glycol, glutaric acid, malic acid, malonic acid, oxalic acid, succinic acid, and adipic acid.

In some embodiments, the implants may be formed of poly-4-hydroxybutyrate (P4HB) and copolymers thereof, or poly(butylene succinate) (PBS) and copolymers thereof. In embodiments, the P4HB and PBS polymers, and copolymers thereof, may not be crosslinked. In embodiments, the PBS polymer and copolymers may further include one or more of the following: branching agent, cross-linking agent, chain extender agent, and reactive blending agent. The PBS and P4HB polymers and copolymers may be isotopically enriched in some embodiments.

In some embodiments, the polymers used to prepare the implants may have weight average molecular weights of 50 to 1,000 kDa, 90 to 600 kDa, and/or from 200 to 450 kDa relative to polystyrene determined by GPC, although other weight average molecular weight polymers are contemplated.

In some embodiments, the prostheses may be formed of degradable materials, including but not limited to, thermoplastic or polymeric degradable materials. Combinations of the foregoing are contemplated. In some embodiments, the prosthesis may be formed of one or more absorbable polymers or copolymers, absorbable thermoplastic polymers and copolymers, and/or absorbable thermoplastic polyesters. The prostheses may be formed of polymers including, but not limited to, polymers of glycolic acid, lactic acid, 1,4-dioxanone, trimethylene carbonate, 3-hydroxybutyric acid, 4-hydroxybutyrate, ε-caprolactone, including polyglycolic acid, polylactic acid, polydioxanone, polycaprolactone, copolymers of glycolic and lactic acids, such as VICRYL® polymer, MAXON® and MONOCRYL® polymers, and including poly(lactide-co-caprolactones); poly(orthoesters); poly anhydrides; poly(phosphazenes); polyhydroxyalkanoates; synthetically or biologically prepared polyesters; polycarbonates; tyrosine polycarbonates; polyamides (including synthetic and natural polyamides, polypeptides, and poly(amino acids)); polyesteramides; poly(alkylene alkylates); polyethers (such as polyethylene glycol, PEG, and polyethylene oxide, PEO); polyvinyl pyrrolidones or PVP; polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyalkylene oxalates; polyalkylene succinates; poly(maleic acids); silk (including recombinant silks and silk derivatives and analogs); chitin; chitosan; modified chitosan; biocompatible polysaccharides; hydrophilic or water soluble polymers, such as polyethylene glycol, (PEG) or polyvinyl pyrrolidone (PVP), with blocks of other biocompatible or biodegradable polymers, for example, poly(lactide), poly(lactide-co-glycolide, or polycaprolactone and copolymers thereof, including random copolymers and block copolymers thereof.

In some embodiments, an implantable prosthesis may be loaded, filled, and/or coated with an appropriate therapeutic composition, including by blending the material composition of the prosthesis with the therapeutic composition and/or fillers. In some embodiments, the fillers may include nanoparticles (e.g., silver nanoparticles) and/or nanotubes (e.g., single walled carbon nanotubes) for antimicrobial properties. Any suitable filler material known in the art to enhance the antimicrobial properties of implants may be employed as the present disclosure is not so limited. This may include coatings, absorbed materials retained in the porous scaffold of the prosthesis, adsorbed materials, compounds functionally bonded to the material of the implantable prosthesis, and/or any other appropriate way of associating a therapeutic composition with the implantable prosthesis. Appropriate types of therapeutic compositions may include, but are not limited to, cells, stem cells, differentiated cells, fat cells, muscle cells, platelets, pedicles, vascular pedicles, tissue masses, extracellular adipose matrix proteins, gels, hydrogels, hyaluronic acid, collagen, bioactive agents, drugs, antibiotics, and other appropriate therapeutic compositions that may be desirable to deliver to the implant site. The cells and tissues, which may be delivered and/or coated or injected into the prostheses, may be autologous. The prostheses may be used for autologous fat transfer. The cells added, coated or injected on the prosthesis may include pancreatic islet cells, hepatic cells, and stem cells genetically altered to contain genes for treatment of patient illnesses. The prostheses may include bioactive agents to stimulate cell in-growth, including growth factors, cell adhesion factors, cellular differentiating factors, cellular recruiting factors, cell receptors, cell-binding factors, cell signaling molecules, such as cytokines, and molecules to promote cell migration, cell division, cell proliferation and extracellular matrix deposition. The prostheses may also be partially or entirely coated and/or contain agents to prevent tissue adhesion, or agents to prevent cell proliferation, particularly to delay cell invasion into the prostheses.

In some embodiments, the implantable prostheses may be partially or entirely loaded, filled, coated, or otherwise incorporated with bioactive agents. Bioactive agents may be included in the prostheses for a variety of reasons. For example, bioactive agents may be included in order to improve tissue in-growth into the implant, to improve tissue maturation, to provide for the delivery of an active agent, to improve wettability of the implant, to prevent infection, and to improve cell attachment. The bioactive agents may also be incorporated into the material composition of the substrate of the subunits, including by blending the material composition and bioactive agents.

The prostheses can contain active agents designed to stimulate cell in-growth, including growth factors, cell adhesion factors including cell adhesion polypeptides, cellular differentiating factors, cellular recruiting factors, cell receptors, cell-binding factors, cell signaling molecules, such as cytokines, and molecules to promote cell migration, cell division, cell proliferation and extracellular matrix deposition. Such active agents include fibroblast growth factor (FGF), transforming growth factor (TGF), platelet derived growth factor (PDGF), epidermal growth factor (EGF), granulocyte-macrophage colony stimulation factor (GMCSF), vascular endothelial growth factor (VEGF), insulin-like growth factor (IGF), hepatocyte growth factor (HGF), interleukin-1-B (IL-1B), interleukin-8 (IL-8), and nerve growth factor (NGF), and combinations thereof. As used herein, the term “cell adhesion polypeptides” refers to compounds having at least two amino acids per molecule that are capable of binding cells via cell surface molecules. The cell adhesion polypeptides include any of the proteins of the extracellular matrix which are known to play a role in cell adhesion, including fibronectin, vitronectin, laminin, elastin, fibrinogen, collagen types I, II, and V, as well as synthetic peptides with similar cell adhesion properties. The cell adhesion polypeptides also include peptides derived from any of the aforementioned proteins, including fragments or sequences containing the binding domains.

In some embodiments, the implantable prostheses may be loaded, filled, coated, or otherwise incorporated with wetting agents designed to improve the wettability of the various surfaces of the prostheses to allow fluids to be easily adsorbed onto the prosthesis surfaces, and to promote cell attachment and or modify the water contact angle of the prosthesis surface. Examples of wetting agents include polymers of ethylene oxide and propylene oxide, such as polyethylene oxide, polypropylene oxide, or copolymers of these, such as PLURONICS®. Other suitable wetting agents may include surfactants or emulsifiers.

In some embodiments, the implantable prostheses may be loaded, filled, coated, or otherwise incorporated with gels, hydrogels or living hydrogel hybrids to further improve wetting properties and to promote cellular growth throughout the prosthesis. Hydrogel hybrids may consist of living cells encapsulated in a biocompatible hydrogel like gelatin, methacrylated gelatin (GelMa), silk gels, and hyaluronic acid (HA) gels.

Other bioactive agents that can be incorporated in the prostheses may include antimicrobial agents, in particular antibiotics, disinfectants, oncological agents, anti-scarring agents, anti-inflammatory agents, anesthetics, small molecule drugs, anti-adhesion agents, inhibitors of cell proliferation, anti-angiogenic factors and pro-angiogenic factors, immunomodulatory agents, and blood clotting agents. The bioactive agents may be proteins such as collagen and antibodies, peptides, polysaccharides such as chitosan, alginate, hyaluronic acid and derivatives thereof, nucleic acid molecules, small molecular weight compounds such as steroids, inorganic materials such as hydroxyapatite and ceramics, or complex mixtures such as platelet rich plasma. Suitable antimicrobial agents include: bacitracin, biguanide, triclosan, gentamicin, minocycline, rifampin, vancomycin, cephalosporins, copper, zinc, silver, and gold. Nucleic acid molecules may include DNA, RNA, siRNA, miRNA, antisense or aptamers.

In some embodiments, the implantable prostheses may be loaded, filled, coated, or otherwise incorporated with allograft material and xenograft materials, including acellular dermal matrix material and small intestinal submucosa (SIS). In an embodiment, the prosthesis may contain a vascular pedicle or other tissue mass. In some embodiments, the prostheses may incorporate systems for the controlled release of the therapeutic or prophylactic agents.

In some embodiments, the implantable prostheses may be loaded, filled, coated, or otherwise incorporated with allograft or xenograft tissue and cells prior to implantation, during implantation, or after implantation, or any combination thereof. In some embodiments, the prostheses may be coated with autologous tissue and cells from the patient prior to implantation, during implantation, or after implantation, or any combination thereof. The autologous tissue and cells may include one or more of the following autologous fat, fat lipoaspirate, fat tissue, injectable fat, adipose tissue, adipose cells, fibroblast cells, and stem cells, including human adipose tissue-derived stem cells, also known as preadipocytes or adipose tissue-derived precursor cells, and fibroblast-like stem cells. In one embodiment, the prostheses may be coated with autologous tissue and cells as described herein, and may also further include a vascular pedicle or other tissue mass. As will be evident herein, the prostheses are designed to create not only a shape for the implant, such as a breast implant, but also a large surface area that can retain the autologous tissue and cells to encourage tissue in-growth.

In some embodiments, the polymers and copolymers composition of the prostheses may have low moisture contents to ensure the prostheses can be produced with prolonged strength retention, and good shelf life. In some embodiments, the polymers and copolymers that are used to prepare the prostheses have a moisture content of less than 1,000 ppm (0.1 wt %), less than 500 ppm (0.05 wt %), less than 300 ppm (0.03 wt %), less than 100 ppm (0.01 wt %), and/or less than 50 ppm (0.005 wt %).

It should be appreciated that the prostheses should have a suitably low endotoxin content prior to implantation. The compositions used to prepare the prostheses may have a low endotoxin content. In some embodiments, the endotoxin content may be low enough so that the prostheses produced from the polymer compositions have an endotoxin content of less than 20 endotoxin units per prosthesis as determined by the limulus amebocyte lysate (LAL) assay. For example, the polymeric compositions used to prepare the prosthesis may have an endotoxin content of <2.5 EU/g of polymer or copolymer. In another example, the P4HB polymer or copolymer, or PBS polymer of copolymer have an endotoxin content of <2.5 EU/g of polymer or copolymer.

In some embodiments, the prostheses of the present disclosure may include one or more markers for external detection of the prosthesis location. For example, a prosthesis may include a radiopaque marker (e.g., a metallic tag, radio opaque materials incorporated into the printed walls of an implant formed with an additive manufacturing process, or other appropriate type of marker), which may be visible and distinct over the nearby anatomy during x-ray imaging. The markers may be formed of any suitable medical materials that may be medically imaged. Medical imaging may be performed using, for example, radiographic imaging modalities (e.g., x-ray imaging), magnetic resonance imaging (MRI), ultrasonography, fluoroscopy, or computed tomography. The marker may therefore be formed of any non-absorbable, biocompatible materials, which may refer to a material that does not cause any adverse reactions to a patient's health and that does not disintegrate over the lifetime of the patient. Non-absorbable, biocompatible materials may include, but are not limited to, metal containing materials, polymer materials, ceramic materials, or composite materials that include metals, polymers, or combinations of metals and polymers. Suitable metals may include, but are not limited to, gold, iridium, nickel, rhodium, silver, tantalum, titanium, stainless steel and alloys thereof, combinations thereof, and/or others. Suitable polymers include, but are not limited to, polyvinyl alcohol, polyurethanes, polyolefins, polyesters, polypropylenes, polyimides, polyetherimides, fluoropolymers, thermoplastic liquid polymers (LCP) such as, for example, Vectra® by Celanese, polyethylether ketones such as, for example, PEEK™ by Vitrex, polyamides, polycarbonates such as, for example, Makrolon® by Bayer Polymers, polysulfones, polyethersufones, polyphenylsulfones such as, for example, Radel® by Rowland Technologies, nylon, nylon copolymers, combinations thereof, and/or others. In some embodiments, the marker may include a shape-memory material, including, but not limited to, nitinol, titanium, or any shape-memory polymers.

Certain additives may be incorporated into the implant, preferably in the polymeric compositions that are used to make the scaffold. In one embodiment, these additives are incorporated with the polymers or copolymers described herein during a compounding process to produce pellets that can be subsequently processed to produce the scaffolds. For example, pellets may be injection molded, extruded, or more preferably printed using an additive manufacturing process, to form scaffolds or unit cells of the scaffolds. In another embodiment, the pellets may be ground to produce powders suitable for further processing, for example, using additive manufacturing processes. Alternatively, powders suitable for further processing, for example using additive manufacturing processes, may be formed directly by blending the additives and polymer or copolymer. If desired, powders used for processing may be sieved to select an optimum particle size range. In another embodiment, the additives may be incorporated into the polymeric compositions used to prepare the scaffolds of the implants using a solution-based process.

In some embodiments, the above noted additives may be nucleating agents and/or plasticizers. These additives may be added to the polymeric compositions used to prepare the scaffolds of the implants in sufficient quantity to produce the desired result. In general, these additives may be added in amounts between 1% and 20% by weight relative to a total weight of the material. Nucleating agents may be incorporated to increase the rate of crystallization of the polymer, copolymer or blend. Such agents may be used, for example, to facilitate fabrication of the scaffold, and to improve the mechanical properties of the scaffold. Preferred nucleating agents include, but are not limited to, salts of organic acids such as calcium citrate, polymers or oligomers of PHA polymers and copolymers, high melting polymers such as PGA, talc, micronized mica, calcium carbonate, ammonium chloride, and aromatic amino acids such as tyrosine and phenylalanine.

Plasticizers that may be incorporated into the polymeric compositions for preparing the scaffolds of the implants include, but are not limited to, di-n-butyl maleate, methyl laureate, dibutyl fumarate, di(2-ethylhexyl) (dioctyl) maleate, paraffin, dodecanol, olive oil, soybean oil, polytetramethylene glycols, methyl oleate, n-propyl oleate, tetrahydrofurfuryl oleate, epoxidized linseed oil, 2-ethyl hexyl epoxytallate, glycerol triacetate, methyl linoleate, dibutyl fumarate, methyl acetyl ricinoleate, acetyl tri (n-butyl) citrate, acetyl triethyl citrate, tri (n-butyl) citrate, triethyl citrate, bis(2-hydroxyethyl)dimerate, butyl ricinoleate, glyceryl tri-(acetyl ricinoleate), methyl ricinoleate, n-butyl acetyl rincinoleate, propylene glycol ricinoleate, diethyl succinate, diisobutyl adipate, dimethyl azelate, di(n-hexyl) azelate, tri-butyl phosphate, and mixtures thereof. Particularly preferred plasticizers are citrate esters.

In some embodiments, the implantable prostheses may be formed using an additive manufacturing technique due to the complex geometry of the subunits. For example, additive manufacturing techniques may be used to construct the implant's flexible scaffold, which allows precise control of the shape of the implant's scaffold. Suitable methods for forming the subunit scaffold include fused filament fabrication, fused pellet deposition, melt extrusion deposition, selective laser melting (e.g., fused powder bed), printing of slurries and solutions using a coagulation bath, printing using a binding solution and granules of powder, stereolithography printing, melt extrusion deposition (MED), and/or other appropriate additive manufacturing processes. In some embodiments, the scaffold of the implant may preferably be prepared by melt extrusion deposition (MED) though any appropriate additive manufacturing process may be used. While specific manufacturing techniques are listed above, it should be appreciated that any fabrication system or method may be employed to form any portion (e.g., scaffold) of the implants described herein, as the present disclosure is not so limited.

In some embodiments, the three-dimensional implants of the present disclosure may include a scaffold as previously described, as well as one or more support structures to locally modify a property of the implant. The support structures may also serve to facilitate a process, such as delivery or implantation. The implants may include support structures such as buttresses, rods, pillars, fibers, paddles, protrusions, pins, dimples, rings, tunnels, tubes, shells, panels, beams (including I-beams, U-beams, W-beams and cylindrical beams), combinations thereof, and/or any other suitable structure to help modify the local properties of the implant. For example, the implant may include pillars extending partially or entirely in one or more directions relative to the implant (e.g., radial, axial), and may have linear, non-linear, curvilinear, combinations thereof, and/or any other suitable structure. In some embodiments, the implant may include one or more growth chambers for cells and tissues. In some embodiments, the implants may include one or more openings to allow the insertion of a vascular pedicle, or other mass of tissue into the implant or so that the implant sandwiches the vascular pedicle or other mass of tissue, as well as one or more openings or passages, including one or more transverse passages, to allow insertion of a vascular pedicle or other tissue mass in the implant.

In some embodiments, the implant may include an external shell or coating may be arranged on at least a portion (or, optionally, entirely around) the exterior surface of the scaffold. The shell may be used to deliver an initial dose of a bioactive agent (or any other material), and/or may serve a mechanical or structural function for the implant. In some embodiments, the thickness of the shell or coating may range between approximately 0.01 mm to 5 mm, between 0.5 mm to 2 mm, between 0.1 mm to 1 mm, and/or any other suitable thickness. In some embodiments, the shell may be formed of concentric stacks of a filament at the periphery of the scaffold. In other embodiments, the thickness of the shell is formed of more than one layer of filaments. In other embodiments still, the shell may be formed of a foam with interconnected pores. In embodiments, the shell is an open cell foam, and/or an open cell foam including poly-4-hydroxybutyrate or copolymer thereof or poly(butylene succinate) or copolymer thereof. In some embodiments, the shell may be heat treated to minimize the roughness of the outer surface of the shell.

In some embodiments, the shell or coating may be permeable to a needle to enable a surgeon to suture or otherwise fixate the implant at the implant site. Alternatively, or in combination with, the implants may include retainers, such as barbs or tacks, on one or more exterior surfaces of the implant so that the implant can be anchored in the body without the use of sutures. The implants may include the retainers in the outlying borders of the implant or scaffold structure of the implant. In embodiments, the retainers may be located on the implant to allow the implant to be anchored to the chest wall.

In some embodiments, the implants may include one or more suture tabs so that the implants can be anchored in the body using sutures or staples. The number of tabs may depend on the implant size (e.g., larger implants may require more tabs). The tabs attached to the implant may have sufficient strength retention in vivo to resist mechanical loads, and to allow sufficient in-growth of tissue into the implant in order to prevent subsequent movement of the implant after implantation. In some embodiments, the suture pullout strength of the tabs attached to the implant, may be greater than 10 N and/or greater than 20 N. It should be appreciated that the implants may be directly fixated in the body without the use of suture tabs or shells, as the present disclosure is not so limited.

In some embodiments, methods of implantation of the implant in the breast of a patient may include at least the steps of: (i) making at least one incision to gain access to the breast tissue of the patient, (ii) separating the skin and subcutaneous fascia from the breast mound of the breast, (iii) positioning the implant on the breast mound of the breast, (iv) securing the implant to the tissue surrounding the breast mound of the breast, and (v) closing the incisions in the breast. In some embodiments, this method further includes one or more of the following steps: (a) preparing a sample of lipoaspirate, and coating or filling the implant with the sample prior to implantation of the implant, (b) preparing a sample of lipoaspirate, and coating or filling the implant with the sample after implantation of the implant, preferably by injecting the sample into the implant, (c) inserting a vascular pedicle into the implant prior to, or after, implantation of the implant, and (d) suturing or stapling the implant in place. The implant may be implanted in a sub-glandular, sub-pectoral or pre-pectoral position. In embodiments, the implant may be sutured to the tissue surrounding the breast mound (e.g., the fascia surrounding the pectoral muscle underlying the breast mound).

As noted previously, in some embodiments, soft tissue implantable prostheses may be used for soft tissue regeneration, augmentation, repair, reinforcement, replacement, and/or reconstruction. For example, the implants may be used to reshape the breast, fill voids in the breast, lift the breast, and augment the breast. The implants can reduce the need to use permanent breast implants during mastectomy, mastopexy, lumpectomy, and breast augmentation procedures. The implants may be biocompatible, and in some instances, may be absorbable such that the implant may be replaced in vivo by the patient's tissue as the implants degrade. The implants may have a compressive modulus that allows the implant to temporarily deform under a compressive force, recover their shape from compression when the force is removed, and have a feel similar to breast tissue. In some embodiments, the implants can be coated or filled with materials to induce tissue ingrowth and/or reduce the risk of infection. For example, the implants may be coated or filled with autologous tissue, autologous fat, fat lipoaspirate, injectable fat, adipose cells, fibroblast cells, and stem cells prior to implantation, during implantation, or post-implantation.

In view of the above, the disclosed implantable prosthesis may be used for a number of different applications, and may offer many different benefits. This may include, for example, soft tissue reconstruction and/or augmentation. This may include reconstruction of surgically excised or resected tissue (e.g., in a lumpectomy procedure), as well as natural soft tissue volume loss. The prosthesis may have mechanical and geometric properties akin to natural tissue to mimic the natural feel of tissue. The prosthesis may further serve as a scaffold for tissue infiltration where tissue may grow into the prosthesis in some embodiments. Tissue in-growth into the void space of the prosthesis may also have an added benefit of an improved cosmetic outcome, resistance to migration and malposition, as well as reduction of risk of capsular contracture. The prosthesis may further serve as an indicator of a biopsy and/or tissue resection site. The prosthesis may be visible using one or more medical imaging systems, to enable external detection of the site for therapeutic and imaging applications. The prosthesis may have the benefit of a reduced clinical target volume in radiotherapy and improve cosmetic outcomes following lumpectomy. However, instances in which different benefits are offered by the systems and methods disclosed herein are also possible.

The implantable prostheses of the present disclosure may be used in any suitable application. In some embodiments, the prostheses may be implanted into soft tissue following a biopsy (and/or any other procedure) during treatment of cancers, such as breast, abdominal, liver, muscle, kidney, lung, and prostate cancer. In some embodiments, the prosthesis may be used in soft tissue reconstruction applications, such that it may serve as a breast implant, breast lift device, breast augmentation device, nipple implant, facial reconstruction device, buttock implant, malar augmentation device, cosmetic repair device, soft tissue regeneration device, hernia implant, hernia plug, wound healing device, tissue engineering scaffold, scaffold for a vascular pedicle or other tissue mass, guided tissue repair/regeneration device, bulking or filling device, void filler, device for treatment of vesicoureteral reflux, cell seeded device, drug delivery device, combinations thereof, and/or any other suitable application. In embodiments, the implant has a shape and size suitable for use in breast surgery procedures, including breast augmentation, breast reconstruction and mastopexy.

“Absorbable” as generally used herein means the material is degraded in the body, and the degradation products are eliminated or excreted from the body. The terms “absorbable”, “resorbable”, “degradable”, and “erodible”, with or without the prefix “bio”, can be used interchangeably herein, to describe materials broken down and gradually absorbed, excreted, or eliminated by the body, whether degradation is due mainly to hydrolysis or mediated by metabolic processes.

“Bioactive agent” as generally used herein refers to therapeutic, prophylactic or diagnostic agents, agents that promote healing and the regeneration of host tissue, and also therapeutic agents that prevent, inhibit or eliminate infection. “Agent” includes a single such agent and is also intended to include a plurality.

“Biocompatible” as generally used herein means the biological response to the material or device being appropriate for the device's intended application in vivo. Any metabolites of these materials should also be biocompatible.

“Blend” as generally used herein means a physical combination of different polymers, as opposed to a copolymer formed of two or more different monomers.

“Compressive modulus” as used herein is measured with a mechanical testing bench (QTest™/1 L, MTS, USA) at a cross-head speed of 20 mm min⁻¹. Samples are preloaded (10% of the initial compressive load) to engage the load and compressed up to 1 mm (20%) of their original height. Clinically relevant cyclic load is repeated 10 times and compressive modulus is calculated based on secondary cyclic load due to the artifact caused by a take up of slack, and alignment or seating of the specimen. Compressive modulus may also be measured using ASTM standards ASTM D1621-16 or ASTM D695-15.

“Compression resilience” as used herein is calculated as the work done during compression recovery divided by the work done during compression multiplied by 100.

“Copolymers of poly-4-hydroxybutyrate” as generally used herein means any polymer containing 4-hydroxybutyrate with one or more different hydroxy acid units. The copolymers may be isotopically enriched.

“Copolymers of poly(butylene succinate)” as generally used herein means any polymer containing 1,4-butanediol and succinic acid units, and one or more different diol or diacid units. The copolymers may include one or more of the following: branching agent, cross-linking agent, chain extender agent, and reactive blending agent. The copolymers may be isotopically enriched.

“Endotoxin content” as generally used herein refers to the amount of endotoxin present in an implant or sample, and is determined by the limulus amebocyte lysate (LAL) assay.

“Infill density” as used herein is the ratio of volume occupied by 3D printed material in an implant scaffold divided by the total volume of the 3D printed scaffold expressed as a percentage.

“Poly(butylene succinate)” as generally used herein means a polymer containing 1,4-butanediol units and succinic acid units. The polymer may include one or more of the following: branching agent, cross-linking agent, chain extender agent, and reactive blending agent. The polymer may be isotopically enriched.

“Poly(butylene succinate) and copolymers” includes polymers and copolymers prepared with one or more of the following: chain extenders, coupling agents, cross-linking agents and branching agents.

“Poly-4-hydroxybutyrate” as generally used herein means a homopolymer containing 4-hydroxybutyrate units. It can be referred to herein as P4HB or TephaFLEX® biomaterial (manufactured by Tepha, Inc., Lexington, Mass.). The polymers may be isotopically enriched.

“Soft tissue” as used herein means body tissue that is not hardened or calcified. Soft tissue excludes hard tissues such as bone and tooth enamel.

“Strength retention” refers to the amount of time that a material maintains a particular mechanical property following implantation into a human or animal. For example, if the tensile strength of a resorbable fiber or strut decreases by half over 3 months when implanted into an animal, the fiber or strut's strength retention at 3 months would be 50%.

“Surface roughness” (Ra) as used herein is the arithmetic average of the absolute values of the profile height deviations from a mean line, recorded within an evaluation length.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

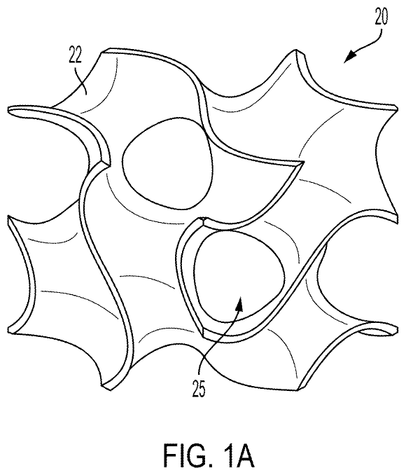

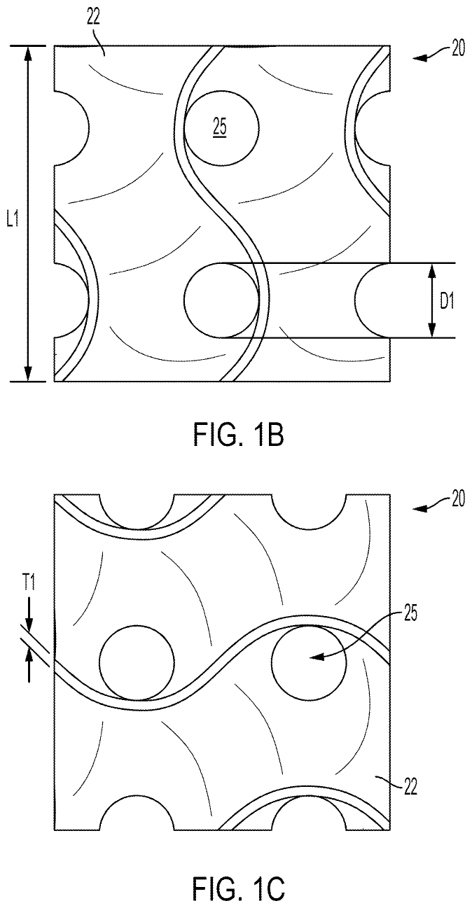

FIG. 1A shows an exemplary subunit 20 of an implant scaffold according to some embodiments. The subunit 20 may include a TPMS or gyroid surface 22 which form macro pores 25 extending through the resulting scaffold to provide an interconnected series of pores extending throughout the implant. As shown in the isometric view of FIG. 1A, the gyroid surface 22 may increase the surface area of the subunit 20 while reducing localized stresses, such that the subunit and the scaffold formed of said subunits may be highly compressible and may recover said compressibility, as described earlier.

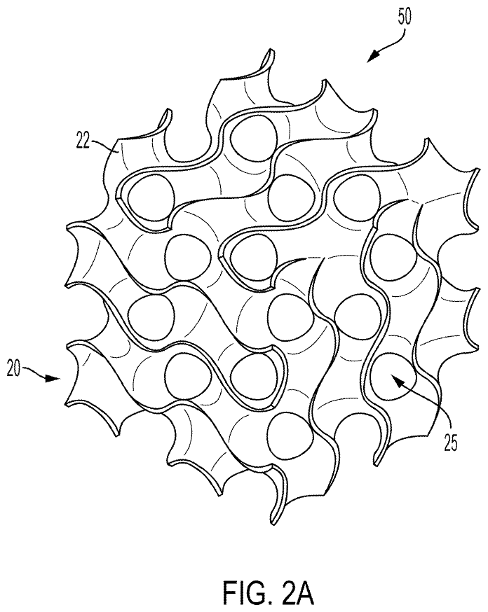

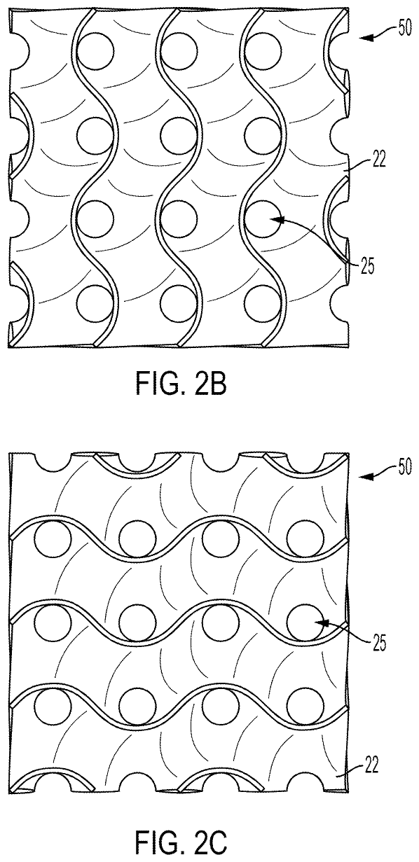

FIGS. 1B-2C show various views of the subunit 20 from FIG. 1A having a gyroid surface 22. The subunit 20 may have a length L1 associated with a single repeating subunit of the scaffold, as shown in FIG. 1B. It should be appreciated that for this exemplary subunit 20, the length may be approximately equal in all three cardinal directions. However, subunits having non-cubic lengths are contemplated. In some embodiments, as discussed above, the greater the length L1 of the subunit, the greater the pore 25 size, such that the density and therefore weight of the implant may be reduced, while the strength or recoverability of the scaffold may be reduced. The length L1 of the subunit may therefore be selected to provide a desired balance between these two parameters depending upon the application and a variety of other parameters of the implant (e.g., constituent material stiffness). Accordingly, the length L1 of the subunit may be any suitable value, including, but not limited to, greater than or equal to approximately 2 mm, 5 mm, 1 cm, 2 cm, 3 cm, and/or any other suitable length. The length L1 may also be less than or equal to 3 cm, 2 cm, 1 cm, 5 mm, 2 mm, and/or any other suitable length. Combinations of the foregoing, including subunit lengths L1 between 2 mm and 3 cm are contemplated, as well as ranges greater than and less than the aforementioned ranges. The repeating subunit is shown to be generally cubic for ease of visualization, and it should be appreciated that the repeating subunit may be any polyhedral or curved three-dimensional shape. The length L1 may be associated with a sidewall length or an average diameter of the repeating subunit, as applicable.

As shown in FIG. 1B, the pores 25 of the subunit 20 may have an average diameter D1. It should be appreciated that in some embodiments, the average pore diameter D1 may scale in some fashion with the length of the subunit L1. For example, a subunit having a length of 1 cm may have an average pore diameter of approximately 2.5 mm, and a subunit having a length of 2 cm may have an average pore diameter of approximately 5 mm, if the wall thickness of the subunit, shown as thickness T1 in FIG. 1C is substantially the same. In other embodiments, the average pore diameter may not scale with the length of the subunit, if other geometric parameters (e.g., wall thickness) are also adjusted. Therefore, the gyroid subunits of the present disclosure may have any suitable average pore diameter, or other appropriate type of maximum transverse dimension depending on pore geometry, desirable for the application (e.g., tissue infiltration and/or degradation rate, compressibility, implant site mechanics), irrespective of the subunit size.

Exemplary and non-limiting average pore diameters D1, or other transverse dimensions, of the subunit may be greater than or equal to approximately 0.05 mm, 0.075 mm, 0.1 mm, 0.5 mm, 1 mm, 2 mm, 5 mm, 1 cm, or other appropriate dimensions. The average pore diameter or other transverse dimension may also be less than or equal to approximately 2 cm, 1 cm, 5 mm, 2 mm, 1 mm, 0.5 mm, 0.1 mm, 0.075 mm, 0.05 mm, and/or any other suitable dimension. Combinations of the foregoing, including average pore diameters or other transverse dimensions between approximately 0.075 mm and 2 cm, 0.5 mm and 1 cm, 1 cm and 5 mm, 0.5 mm and 2 cm are contemplated, as well as ranges greater than and less than the aforementioned ranges.

The wall thickness T1 of the subunit 20, as shown in FIG. 1C, may be determined based at least in part on a variety of parameters, including desired compressibility and strength of the implant, degradation rate (if applicable, based on material composition), as well as the fabrication method. For example, if the scaffold is formed using an MED technique, the minimum wall thickness may be determined by the minimum possible deposited filament diameter. In some embodiments, the filament diameter may correspond to the nozzle diameter of the system. However, the filament diameter may be greater than or less than the nozzle diameter, dependent upon scanning speed. In some embodiments, the wall thickness T1 may correspond to a single filament diameter. In other embodiments, more than one filament may be used to form a thicker wall thickness.

Exemplary and non-limiting wall thicknesses T1 of the subunit, may be greater than or equal to approximately 0.01 mm, 0.05 mm, 0.1 mm, 0.15 mm, 0.2 mm, 0.25 mm, 0.3 mm, 0.4 mm, 0.5 mm, 1 cm, and/or less than or equal to approximately 1 cm, 0.5 mm, 0.4 mm, 0.3 mm, 0.25 mm, 0.2 mm, 0.15 mm, 0.1 mm, 0.05 mm, 0.01 mm, and/or any other suitable thickness. Combinations of the foregoing, including wall thicknesses between 0.01 mm and 1 cm are contemplated, as well as ranges greater than and less than the aforementioned ranges.

FIGS. 2A-2C show various views of one embodiment of a partial scaffold 50 formed of 8 subunits 20, similar to those shown in FIGS. 1A-1C. The scaffold may have interconnected macro pores 25 formed between the gyroid surfaces of the subunits. It should be appreciated that although 8 subunits are shown in the partial exemplary scaffold of FIGS. 2A-2C, any suitable number of subunits 20 may be employed, greater than and less than 8 subunits, dependent upon the application of the implant.

The gyroid TPMS of the subunits shown in FIGS. 1A-2C may behave substantially isotropic in more than one direction, such that their mechanical properties in more than one direction may be substantially equivalent. Such behavior may more accurately mimic natural tissue, while also enabling the implant to be delivered through a small incision site without significant risk of damage or discomfort to the patient.

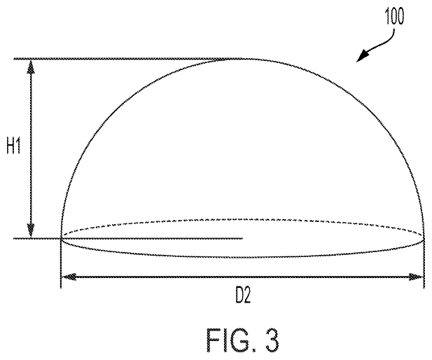

FIG. 3 shows an exemplary breast implant 100 according to some embodiments, the implant may be formed of the TPMS scaffold shown in FIGS. 1A-2C. The implant may be sized and shaped to fit an anatomical site. For example, the implant may be dome-shaped as shown in FIG. 3. It should be appreciated that although the dome is shown to be symmetric, non-symmetric shapes that more accurately mimic the asymmetry of natural tissue are contemplated, as described in greater detail above.

In the depicted embodiment, the implant 100 may have an average base diameter D2 which may be wide enough to span the width of a reconstructed or augmented breast. The average base diameter D2 may be specific to the patient, and thus may be any suitable size, including, but not limited to, greater than or equal to approximately 6 cm, 8 cm, 10 cm, 12 cm, 15 cm, 18 cm, 20 cm, 22 cm, and/or less than or equal to approximately 22 cm, 20 cm, 18 cm, 15 cm, 12 cm, 10 cm, 8 cm, 6 cm, and/or any other suitable average diameter. Combinations of the foregoing ranges, including average base diameters between approximately 6 cm and 22 cm, between 8 cm and 18 cm, and/or ranges greater than and less than the aforementioned ranges are contemplated.

It should be appreciated that in other applications (e.g., lumpectomies), the average diameter of the implant may be smaller to correspond to the resection site size. Accordingly, the implants of the present disclosure are not limited by their shape and/or size.

The implant 100 may also have an average projection height H1, as shown in FIG. 3. Similar to the base diameter D2, the projection height H1 may be customized to the patient, and may therefore be any suitable size, including, but not limited to, between approximately 2 and 15 cm, 3 and 10 cm, 4 and 7 cm, combinations thereof, and/or any other suitable size.

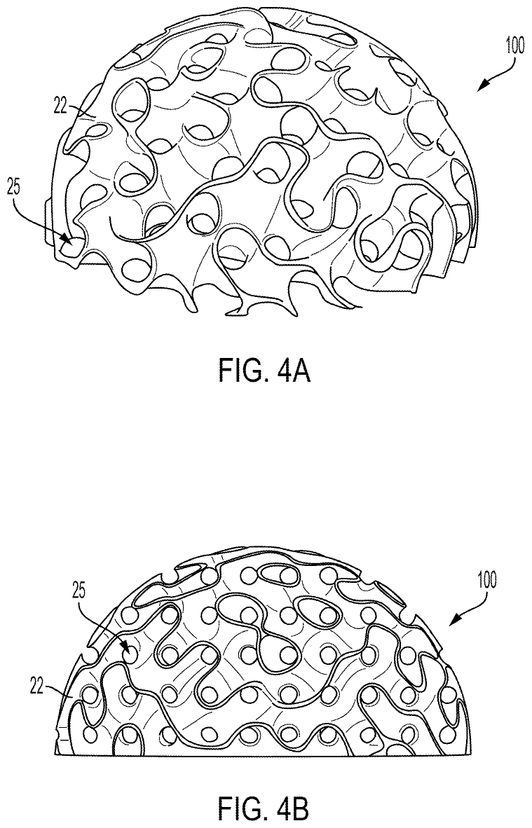

FIGS. 4A-4B show different views of one embodiment of a breast implant scaffold formed of gyroid subunits. The implants 100 are designed with average base diameters of 11 cm and projection heights of 5 cm. The gyroid subunits have interconnected macro pores 25 formed by the gyroid surfaces 22 such that the scaffold may have open porosity. As shown in FIGS. 4A-4B, partial subunits may be employed when the scaffold shape is different from the subunit shape (e.g., dome scaffold vs. cubic subunit). Thus, the implants of the present disclosure may include partial subunits to form the desired scaffold size.

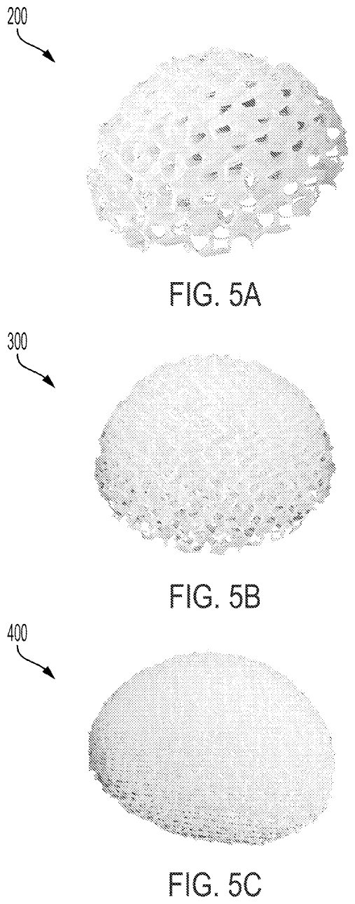

FIGS. 5A-5C show three flexible breast implants with gyroid subunits fabricated using the designs similar to those shown in FIGS. 4A-4B. Specifically, the depicted implants were formed with gyroid subunits with different spatial frequencies with the implant of FIG. 5A formed with the smallest spatial frequency (i.e., largest subunit size) and FIG. 5C formed with the largest spatial frequency (i.e., smallest subunit size). Without wishing to be bound by theory, the spatial frequency of the subunit may be scaled with the infill density such that larger spatial frequencies (i.e., smaller subunit sizes) may be associated with larger infill densities. For example, FIG. 5A shows an implant 200 with a 3% infill density, FIG. 5B shows an implant 300 with a 5% infill density, and FIG. 5C shows an implant 400 with an 8% infill density. As shown in the figures, the gyroid subunit size may scale with the infill density, such that a lower infill density may result in larger subunits (e.g., FIG. 5A) compared to a higher infill density subunit size (e.g., FIG. 5C).

The implants of FIGS. 5A-5C, formed of P4HB were formed using MED. In some exemplary embodiments, pellets of poly-4-hydroxybutyrate (P4HB) (Tepha, Inc., Lexington, MA, Mw 450 kDa) may be loaded into the hopper of an MED based 3D printer including a horizontal extruder feeding into a vertical extruder fitted with a vertical plunger, and a movable stage. An exemplary process for using melt extrusion to eject a series of droplets on demand in preparing an implant is described in Patent Publication No. 2019/0375149, filed Jun. 11, 2019, and entitled “METHODS FOR 3D PRINTING OF POLY-4 HYDROXYBUTYRATE AND COPOLYMERS”, incorporated herein by reference in its entirety.

Pellets of P4HB may have an average diameter of 3.5 mm, moisture content of less than 100 ppm, and may be kept dry in the hopper using a purge of air dried through a silica bed. The temperature profile of the horizontal extruder may be set to 12-14° C. in the build chamber; 110° C. in the first transition zone, 135° C. in second transition zone; and 185° C. in the extrusion zone. The residence time of the polymer in the MED horizontal extruder may be 22 min/cm³. The back pressure may be set to 50 bars (5 MPa). The diameter of the nozzle orifice of the vertical extruder may be 0.15 mm and the drop printing frequency may be 240 drops/sec for the in-fill. Additional exemplary settings for the 3D printer are shown in Table 1.

TABLE 1
Exemplary parameters for MED Printing
of Compressible P4HB Breast Implants

| | |
|---|---|
| Print head temp (° C.) | 185 |
| Barrel zone 2 (° C.) | 135 |
| Barrel zone 1 (° C.) | 110 |
| Build chamber temp (° C.) | 12-14 |
| Screw speed (m/min) | 4 |
| Back pressure (MPa) | 5 |
| Recovery stroke (mm) | 6 |
| Deco speed (mm/s) | 2 |
| Deco stroke (mm) | 4 |
| Discharge nr (%): | 55 |
| In Filling density Body (%) | 3-8 |
| Drop ratio | 1-1.3 |

Table 2 below outlines various measured properties of the three 3D printed compressible breast implants formed of P4HB material and gyroid subunits of FIGS. 5A-5C.

TABLE 2
Properties of Implants with a Porous Gyroid Design

| | | | Weight | | | |
|---|---|---|---|---|---|---|
| | | | per | Surface | | Base |
| Implant | Weight | Volume | Volume | Area | Height | Diameter |
| Infill | (g) | (cm³) | (g/cm³) | (cm²) | (cm) | (cm) |
| Gyroid with | 9.12 | 289 | 0.03 | 1026 | 5.1 | 11.1 |
| 3% Infill | | | | | | |
| (FIG. 5A) | | | | | | |
| Gyroid with | 12.45 | 290 | 0.04 | 1608 | 5.0 | 11.2 |
| 5% Infill | | | | | | |
| (FIG. 5B) | | | | | | |
| Gyroid with | 16.22 | 310 | 0.05 | 2431 | 5.1 | 10.9 |
| 8% Infill | | | | | | |
| (FIG. 5C) | | | | | | |

The three implants of FIGS. 5A-5C underwent cyclic compression testing to mechanically evaluate the implants. The average maximum compressive load at 50% strain, stiffness at 20-40% and 40-50% strain, and dimensional height recovery after cyclical loading are shown in Table 3. The results show that the implants recover greater than 97% of their dimensional height (projection from base of the implant to apex) after cyclical compression.

TABLE 3
Cyclic Compression in the Axial Direction of Implants with a Porous Gyroid Design

| | Avg Max | Stiffness | Stiffness | Dimensional (Height) |
|---|---|---|---|---|
| Implant | Compressive Load | (N/cm) @ 20- | (N/cm) @ 40- | Recovery Post |
| Infill | (N) @ 50% Strain | 40% Strain | 50% Strain | Cyclical Loading |
| Gyroid with | 17.3 | 6.4 | 9.0 | 97 |
| 3% Infill | | | | |
| (FIG. 5A) | | | | |
| Gyroid with | 43.4 | 19.0 | 28.2 | 100 |
| 5% Infill | | | | |
| (FIG. 5B) | | | | |
| Gyroid with | 26.5 | 11.8 | 20.0 | 99 |
| 8% Infill | | | | |
| (FIG. 5C) | | | | |

The breast implants of FIGS. 5A-5B were also passed through a Keller funnel and retained their shape and volume, suggesting that the breast implants may be delivered through a small inlet into the implant site without significant damage or deformation.

FIGS. 5A-5C depict implants which are formed entirely from a gyroid scaffold having substantially uniform properties across the implants. However, implants having regions of varying scaffolds are contemplated.

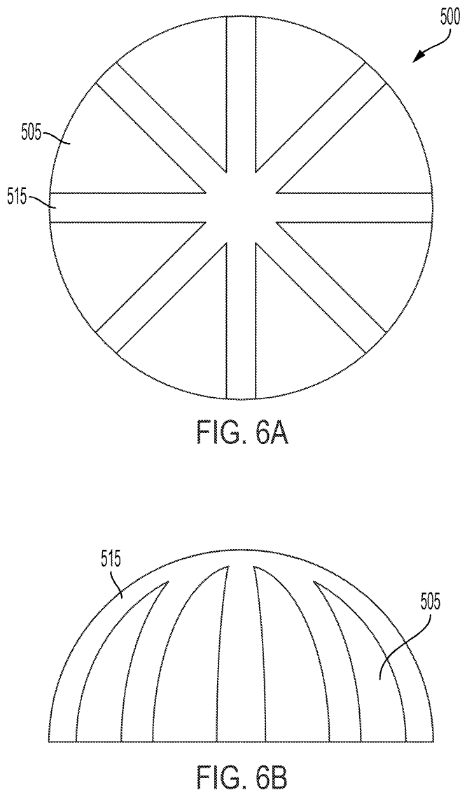

FIGS. 6A-6B depict a breast implant 500 according to some embodiments. The breast implant may include a first TPMS scaffold 505 and a second scaffold 515 arranged along radially emanating lines from the center of the implant 500. The second scaffold may include a TPMS scaffold with subunits different from the first scaffold. For example, the second scaffold 515 may be formed of subunits with a characteristic dimension that is greater than a characteristic dimension of the first scaffold 505, which may facilitate the radial compression of the implant. For example, a spatial frequency of the first scaffold may be less than a spatial frequency of the second scaffold such that the second scaffold may be more dense, and thus, more rigid. In some embodiments, the second scaffold may be arranged on the outer surface of the first scaffold, whereas in other embodiments, the second scaffold may extend through the body of the first scaffold. For example, FIG. 6A may represent a top view of the implant, as well as a scaled cross-sectional view of the implant taken in the axial direction. FIG. 6B may represent a side view of the implant, as well as cross-sectional views of the implant taken along the radial direction. It should be appreciated that more than one scaffold type may be employed in any suitable arrangement to facilitate a desired mechanical and tissue infiltration profile.

It should also be appreciated that although the scaffolds 505, 515 are shown to be distinct, embodiments with a smooth gradient transition between the scaffold regions are contemplated.

In some embodiments, the second scaffold 515 of FIGS. 6A-6B may represent support structures, such as pillars, extending across the surface of the scaffold 505 to provide local mechanical support. In some embodiments, the support structure formed by the second scaffold 515 may serve to deliver a bioactive agent. It should be appreciated that the arrangement of the regions shown in FIGS. 6A-6B is exemplary only, and that other arrangements of secondary scaffolds and/or support structures are contemplated.

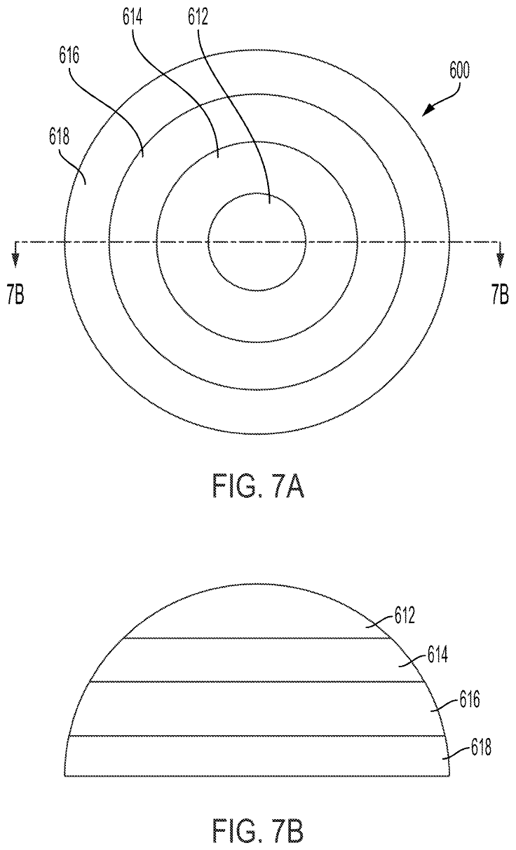

FIGS. 7A-7B depict a breast implant 600 according to other embodiments. The breast implant 600 may be formed of multiple layers 612, 614, 616, 618 of scaffolds that are disposed on one another in a stacked configuration relative to an axial direction of the implant. Each of these layers may be different from one another to provide a desired mechanical behavior. As shown in the cross-sectional view of FIG. 7B taken along line 7B-7B of the implant in FIG. 7A, the layers may extend through the body of the implant 600. In some embodiments, layer 618 may function as a base of the implant. As described relative to FIGS. 6A-6B, the various layers may be distinct or may be arranged as a gradient through the implant. It should also be appreciated that the four layers shown in FIGS. 7A-7B are exemplary only, and that other arrangements of scaffold layers are contemplated.

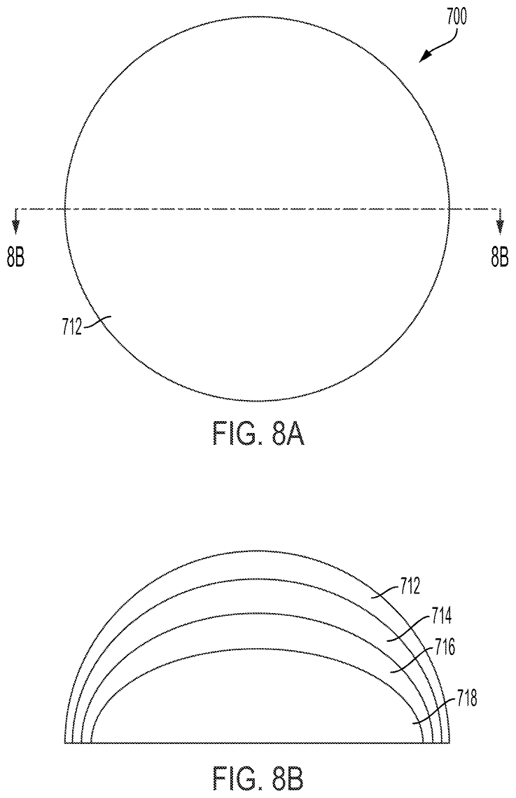

FIGS. 8A-8B depict a breast implant 700 having multiple concentric scaffolds 712, 714, 716, 718 that are disposed on and at least partially surround an underlying layer with layer 718 being the bottom most layer of the implant. FIG. 8B shows a cross-sectional view of the implant taken along line 8B-8B of FIG. 8A. In some embodiments, the outermost layer 712 may serve as a shell or a coating, as previously described, although shell-less implants are contemplated. It should also be appreciated that the four layers shown in FIGS. 8A-8B is exemplary only, and that other arrangements of scaffold layers are contemplated.

Each of the layers or regions shown in FIGS. 6A-8B may represent differences in subunit type, including different materials, spatial frequencies, infill densities, subunit types, and/or other differences that provide different properties for the different portions of the implant. These regional differences in properties may further be influenced by geometric differences in the layers or regions, such as differences in thicknesses for each region or layer. As noted previously, these region-by-region variations in subunit properties and regional geometries may provide differences in stiffness, tissue infiltration properties, capacities for therapeutic compositions, surface roughness, and/or any other desired characteristic among the regions. Furthermore, these variations, in the aggregate, may influence the properties of the overall implant. Thus, it should be appreciated that any combination of the layers or regions (e.g., scaffold regions or support structures, such as those of FIGS. 6A-8B) may be employed to achieve desirable mechanical behavior, tissue infiltration behavior, degradation behavior, or physical characteristic for either a particular region, or for the implant as a whole.

For example, as noted above, in some embodiments, it may be desirable for an implant to have a lower compressive stiffness in a radial direction (e.g., to allow the base to be compressed when passing through a funnel or incision), and a greater compressive stiffness in an axial direction (e.g., to maintain a desired shape over time, and/or to mimic the feel of natural tissue). Additionally or alternatively, it may be desirable in some embodiments for an outer portion of the implant to feel smooth to the touch. In some embodiments, it may be possible to reduce the presence of sharp edges and/or an overall surface roughness of the exterior surface of the implant, for example by providing a shorter distance between adjacent edges of the scaffold and/or using different subunit geometries with alternative geometries. This may beneficially improve handling of the system during implantation as well as other possible benefits. Additionally or alternatively, it may be desirable in some embodiments for the implant to facilitate tissue infiltration (e.g., by providing low infill density, large pore size, etc.). The inventors have recognized and appreciated that such combinations of desirable properties for the implant may be achieved by providing regions or layers of the implant with particular properties which, in the aggregate, produce the desirable properties in the overall implant.

In the exemplary embodiment of FIGS. 8A-8B, the properties of individual layers 712-718 may be selected to achieve these and/or other desirable properties in the implant 700. For example, the core layer 718 may be formed with a relatively low infill density and/or a relatively large pore size. In some embodiments, the core layer may include a TPMS or gyroidal scaffold with an infill density that may optionally be between approximately 4% and 6%. This may facilitate a relatively low compressive stiffness in the radial direction as compared to a higher infill density or smaller pore size. Additionally or alternatively, such a low-density core may facilitate tissue infiltration, as discussed above.

Further, in some embodiments, a first intermediate layer 716 and a second intermediate layer 714, each of which may be concentric with and/or may at least partially surround the core layer 718 as shown in FIG. 8B, may have infill densities which are higher than in the core layer 718 and/or pore sizes that are smaller than in the core layer 718. This may contribute to a higher compressive stiffness in the axial direction, while allowing the radial compressive stiffness to remain in the desired range. In some embodiments, the infill density may increase from the first intermediate layer 716 to the second intermediate layer 714, with the first intermediate layer 716 disposed between the core layer 718 and second intermediate layer 714. In one possible embodiment, the first intermediate layer 716 may include a TPMS or gyroidal scaffold with an infill density of approximately 8%, and the second intermediate layer 714 may include a TPMS or gyroidal scaffold with an infill density of approximately 12%. Such increases may occur discretely or using gradients in the scaffold properties, as described above. The thicknesses of the intermediate layers 716, 714 may further be selected to achieve the desired stiffnesses. For example, in some embodiments, the first intermediate layer 716 may have a thickness of approximately 5 mm to 10 mm, while the second intermediate layer may have a thickness of approximately 1 mm and 3 mm.

Although the embodiment shown includes two intermediate layers, it will be appreciated that only a single intermediate layer may be present in some embodiments. For example, some embodiments may include a single intermediate layer with an infill density of approximately between 8% and 12%, or any other appropriate infill density, and a thickness of approximately 2 mm to 10 mm, 5 mm to 8 mm, or any other appropriate thickness to achieve a desired property in the layer or in the implant. Alternatively, more than two intermediate layers may be present, or there may be a continuous gradient in infill density from a core layer to a shell layer, such that distinct intermediate layers may not be distinguishable.

Further, in some embodiments, an outer layer 712, which may be concentric with and/or may at least partially surround one or more intermediate layers 714, 716 and/or a core layer 718 as shown in FIG. 8B, and may have a greater infill density as compared to the intermediate layer(s) and/or core layer. The outer layer 712 may be formed to provide a desired property such as smoothness, therapeutic compound loading, and/or other appropriate parameter for the outer surface of the implant, for example by utilizing a higher infill density and/or a different subunit type. In some embodiments, a subunit type having a polygonal or honeycomb geometry, such as a hexagonal or octagonal geometry, or in a tessellation pattern such as a Voronoi pattern or others, may provide a smoother feel or other desired property as compared to a TPMS or gyroidal subunit type. Accordingly, in some embodiments, an outer layer may be formed having a Voronoi subunit type, and may have an infill density of approximately 20% to 24% or optionally approximately 22%. Furthermore, in some embodiments, an outer layer may have a relatively small thickness, sufficient to achieve the desired surface feel without substantially impacting a desired stiffness in one or more directions. For example, the outer layer 712 may have a thickness of approximately 0.1 mm to 1.0 mm or approximately 0.5 mm.

It should be appreciated that, although the embodiments above illustrate examples in which infill densities, subunit types, and layer thicknesses are varied throughout different regions to achieve desirable properties of the implant, variations among regions are not limited to infill densities, subunit types, and layer thicknesses. As noted above, other properties which can vary by region include material types, material properties, spatial frequencies, pore sizes, and others. Further, although embodiments in which an exterior region has a greater infill density than an interior region have been described above, it will be appreciated that embodiments in which an interior region has a lower infill density than one or more intermediate/outer regions are also contemplated, as are embodiments in which infill densities in different regions are substantially uniform (e.g., where regions may be defined by differences in pore size, spatial frequency, subunit type, or other properties).

### Example: Rabbit Model Testing

Three groups of the implants described above were experimentally tested by implantation into rabbits. All three groups were substantially dome-shaped, and included a first TPMS scaffold with infill densities that varied among the groups, and a second TPMS scaffold with an infill density that was 11% in all groups. The second scaffold was arranged along four radially emanating lines from the center of the implant, the four lines arranged at right angles to form the shape of a plus sign when viewed from an axial direction. The infill densities of the first scaffolds were approximately 4.5%, approximately 6.7%, and approximately 9.0% in the first, second, and third group, respectively. All implants were 3D printed using P4HB. Implants from each group were removed following 4 weeks, 12 weeks, and 26 weeks of implantation. After removal, implants were evaluated for: material degradation (using scanning electron microscopy (SEM) and by evaluating changes in molecular weight); dimensional stability (by measuring the heights and the base diameters); tissue infiltration (using histological evaluations and computed tomography (CT) scans); and compressive stiffness (both before and after removal of infiltrated tissue).

All three groups exhibited similar material degradation. Scanning electron micrograph (SEM) evaluations revealed significant surface erosion and pitting in all three groups, and all three groups retained about 84% of their molecular weight after 4 weeks, about 56-57% after 12 weeks, and about 27% after 26 weeks.

The rate of tissue infiltration was greatest in the 4.5% group, and lowest in the 9.0% group, though all groups exhibited favorable tissue infiltration behavior. At the 4-week timepoint, loose fibrovascular tissue had formed in all groups. At the 12-week timepoint, soft mature, fibrovascular tissue developed in all groups with adipose tissue developing throughout the 4.5% group and at the periphery of the 9.0% group.

As noted above, compressive stiffness was tested with the infiltrated tissue still present on the implant (i.e., in an “as received” state), and with the infiltrated tissue removed through an enzymatic process (i.e., in a “digested” state). All three groups were also evaluated for compressive stiffness prior to implantation, with the pre-implantation stiffness of the 4.5% group being about 235 kPa, the 6.7% group being about 354 kPa, and the 9.0% group being about 449 kPa.

All three groups exhibited higher compressive stiffness following implantation and tissue infiltration. The stiffness of the 4.5% group in the as received state increased by about 83% at the 4-week timepoint, and then declined at the 12- and 26-week timepoints. The stiffness in the as received state at 26 weeks was approximately 53% higher than the pre-implantation stiffness. The stiffness of the 6.7% group in the as received state increased by about 66% at the 4-week timepoint. At 12 weeks, the stiffness had increased to about 207% higher than the pre-implantation stiffness. At 26 weeks, the 6.7% group's as received stiffness had decreased from the 12-week stiffness, but still exceeded the pre-implantation stiffness by about 86%. The stiffness of the 9.0% group in the as received state had increased by about 55% at 4 weeks, and was about 161% higher than the pre-implantation stiffness at 12 weeks. At 26 weeks, the 9.0% group's as received stiffness was about 152% higher than the pre-implantation stiffness. In all groups, the stiffness in the digested state declined over time, consistent with the material degradation results indicating that the material was being degraded and/or absorbed.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

1. An implantable soft tissue repair prosthesis comprising:
a flexible porous scaffold formed of at least one triply periodic minimal surface, wherein the flexible porous scaffold is configured to recover at least 75% of its original volume upon a 50% compression in at least one direction.

2. The prosthesis of claim 1, wherein an infill density of the flexible porous scaffold is less than approximately 20%.

3. (canceled)

4. The prosthesis of claim 1, wherein an average pore diameter of at least a portion of the flexible porous scaffold is between approximately 0.075 mm to 20 mm.

5.-7. (canceled)

8. The prosthesis of claim 1, wherein the flexible porous scaffold is a first flexible porous scaffold, wherein the prosthesis further comprises a second flexible porous scaffold, and wherein an infill density of the first flexible porous scaffold is different from an infill density of the second flexible porous scaffold.

9. The prosthesis of claim 8, wherein the second flexible porous scaffold is arranged in one or more intermediate layers, the one or more intermediate layers at least partially surrounding a core layer comprising the first flexible porous scaffold, the prosthesis further including a shell layer comprising a third flexible porous scaffold, the shell layer at least partially surrounding the one or more intermediate layers.

10. The prosthesis of claim 9, wherein the infill density of the second flexible porous scaffold and an infill density of the third flexible porous scaffold are each greater than the infill density of the first flexible porous scaffold.

11. The prosthesis of claim 10, wherein the third flexible porous scaffold is formed with a different subunit than at least one of the second flexible porous scaffold and the first flexible porous scaffold.

12. The prosthesis of claim 11, wherein the third flexible porous scaffold is formed in a tessellation pattern.

13. (canceled)

14. The prosthesis of claim 8, wherein the second flexible porous scaffold comprises at least one selected from the group of:
a) a shell,
b) a base,
c) a plurality of pillars, and/or
d) one or more openings for insertion of a vascular pedicle, or other tissue mass.

15. The prosthesis of claim 14, wherein the second flexible porous scaffold comprises the plurality of pillars, and wherein the plurality of pillars includes at least one curvilinear pillar.

16. The prosthesis of claim 8, wherein the infill density of the first flexible porous scaffold is between approximately 3% and 10%, and wherein the infill density of the second flexible porous scaffold is between 5% and 10%.

17. The prosthesis of claim 1, wherein the flexible porous scaffold is a first flexible porous scaffold, wherein the prosthesis further comprises a second flexible porous scaffold, and wherein a degradation profile of the first flexible porous scaffold is different from a degradation profile of the second flexible porous scaffold.

18. The prosthesis of claim 8, further comprising a transition between the first flexible porous scaffold and the second flexible porous scaffold.

19. (canceled)

20. The prosthesis of claim 1, wherein a compressive stiffness of the implant in a first direction is greater than a compressive stiffness of the implant in a second direction.

21. The prosthesis of claim 1, wherein a density of the implant is between 0.02 and 0.2 g/cm³.

22. The prosthesis of claim 1, wherein the flexible porous scaffold is formed of an absorbable polymer comprised of one or more monomers selected from the group: glycolide, lactide, glycolic acid, lactic acid, 1,4-dioxanone, trimethylene carbonate, 3-hydroxybutyric acid, 3-hydroxybutyrate, 4-hydroxybutyric acid, 4-hydroxybutyrate, &-caprolactone, 1,4-butanediol, 1,3-propane diol, ethylene glycol, glutaric acid, malic acid, malonic acid, oxalic acid, succinic acid, and adipic acid.

23. (canceled)

24. The prosthesis of claim 1, wherein the flexible porous scaffold is formed of poly-4-hydroxybutyrate.

25. The prosthesis of claim 1, wherein the triply periodic minimal surface is a gyroid.

26-27. (canceled)

28. A method of implanting an implantable soft tissue prosthesis, the method comprising:
compressing the implantable prosthesis by at least 50% in at least one direction, the implantable prosthesis comprising a flexible porous scaffold formed of at least one triply periodic minimal surface;
delivering the implantable prosthesis to an implant site; and
recovering at least 90% of the compression in the at least one direction.

29. The method of claim 28, further comprising (i) making at least one incision to gain access to the breast tissue of the patient, (ii) separating the skin and subcutaneous fascia from the breast mound of the breast, (iii) positioning the implant sub-glandular, sub-pectoral, or subfascial (iv) securing the implant to nearby tissue, and (v) closing the incisions in the breast.

30. The method of claim 28, further comprising passing the implant through an implant delivery funnel arranged at the implant site.

31. The method of claim 28, further comprising forming the prosthesis using an additive manufacturing process including at least one of: melt extrusion deposition, fused filament fabrication, fused pellet deposition, melt extrusion deposition, selective laser melting, printing of slurries and solutions using a coagulation bath, and printing using a binding solution and polymer granules.

32. The method of claim 28, wherein an infill density of the flexible porous scaffold is less than approximately 20%.

33. The method of claim 28, wherein the flexible porous scaffold is formed of an absorbable polymer.

34. (canceled)

35. The method of claim 28, wherein the triply periodic minimal surface is a gyroid.

36. (canceled)
